(19) United States
(12) Patent Application Publication
(10) Pub. No.: US 2011/0184194 A1
(43) Pub. Date: Jul. 28, 2011

(54) Surfactant Compositions and Synthesis

(76) Inventor: Volker Berl, New York, NY (US)

(21) Appl. No.: 13/007,810

(22) Filed: Jan. 17, 2011

Related U.S. Application Data
(63) Continuation-in-part of application No. 12/958,288, filed on Dec. 1, 2010.
(60) Provisional application No. 61/265,615, filed on Dec. 1, 2009.

(51) Int. Cl. C07D 311/72

(52) U.S. Cl. 549/410

(57) ABSTRACT

Disclosed herein are environmentally benign surfactants including TPGS-550-M, TPGS-750-M and TPGS-1000-M that comprises of diesters composed of racemic α-tocopherol, MPEG-550, MPEG-750 and MPEG-1000, respectively, and a succinic acid fragment. Also disclosed are novel and efficient methods for their synthesis. The surfactants are designed as an effective nanomicelle-forming species for dissolution of hydrophobic compounds and composition and for general use in metal-catalyzed cross-coupling reactions in water.

### RELATED APPLICATION

This application claims the benefit of U.S. Nonprovisional application Ser. No. 12/958,288 filed Dec. 1, 2010, which claims priority to U.S. Provisional Application No. 61/265,615, filed Dec. 1, 2009, both of which are incorporated herein by reference.

### BACKGROUND OF THE INVENTION

Surfactants have been used to prepare stabilized formulations comprising food, beverage, pharmaceutical or nutraceutical products containing nutritional products. Surfactants such as TPGS (polyoxyethanyl-alpha-tocopheryl succinate) and TPGS-1000 (D-alpha-tocopheryl polyethylene glycol 1000 succinate) have been used as solubilizing agents for such stabilized formulations, such as water-soluble formulations including natural omega-fatty acids or non-natural omega-fatty acids. In addition, surfactants, such as PTS (1; FIG. 1), have also been used effectively for organometallic catalyzed reactions, such as Pd- and Ru-catalyzed reactions, that may be performed in water and at room temperature. Name reactions such as Heck, Suzuki-Miyaura and Sonogashira couplings may be carried out in ≦5 wt % PTS/water at room temperature. Other Pd-catalyzed reactions that successfully employ surfactants in water include aminations of aryl halides, allylic aminations of alcohols, and silylations of allylic ethers. Several types of Ru-catalyzed metathesis reactions, including cross- and ring-closing, were shown to be quite amenable to this medium. Such reactions using these surfactants provide products with improved impurity profiles, mild reaction conditions, and thus, result in minimal environmental impact.

We have shown that amphiphile “TPGS-750-M” (2) possesses several important advantages over other known surfactants, such as PTS and TPGS (TPGS-1000), as TPGS-750-M provides better rates of couplings and higher levels of conversion and resulting yields. The 750-M is the monomethylated polyethylene glycol, or “MPEG”, rather than the corresponding PEG diol, as found in PTS and TPGS.

The foregoing examples of the related art and limitations are intended to be illustrative and not exclusive. Other limitations of the related art will become apparent to those of skill in the art upon a reading of the specification and a study of the drawings or figures as provided herein.

### SUMMARY OF THE INVENTION

The present inventor has identified a need for novel and effective surfactants and novel methods for the preparation of the surfactants. In particular, the present application discloses a new combination within the TPGS series of surfactants, namely those using racemic α-tocopherol (written alternatively as DL-α-tocopherol), together with MPEG (rather than PEG), both linked as esters to succinic acid, as new compounds that afford opportunities for multiple uses. In one aspect, a particular advantage of the present TPGS series of surfactants, including TPGS-550-M, TPGS-750-M and TPGS-1000-M, is that each employs a succinic acid linker that is based on relatively inexpensive raw material such as succinic anhydride or succinic acid. In addition, the present application discloses a novel and expedient synthesis of the surfactants that employs racemic α-tocopherol that provides significant economic advantages over the components required for the preparation of nonracemic TPGS-1000 that relies on natural vitamin E, as currently used since the introduction of TPGS by Kodak in the 1950s.

These large number of applications for using the new surfactants as described herein, include, most notably, the solubilization of nutraceuticals. Also of value are applications to pharmaceuticals, cosmetics and cosmeceuticals in water (or saline solution). These uses are in addition to their applications to green chemistry, where they enable solubilization of substrates, reagents, and catalysts, thereby leading to micellar catalysis in water as the only medium, mainly at ambient temperatures.

Accordingly, the present application discloses a novel and efficient synthesis for the preparation of TPGS-MPEG, including TPGS-550-M, TPGS-750-M and TPGS-1000-M. TPGS-750-M, for example, possesses racemic α-tocopherol as its main lipophilic component, and has a relatively inexpensive diester succinic acid linker that is appended to an MPEG chain. The novel synthesis typically employs, although is not limited to, either an MPEG chain that is a 550-M, 750-M, or a 1000-M. For synthetic purposes, use of a monomethylated polyethylene glycol, or “MPEG”, is a key modification en route to these new surfactants, as it obviates the commonly observed, undesired double-ended, diesterification that is problematic when a PEG diol is used, as in the preparation of PTS.

Representative synthetic approaches to TPGS-MPEG, as disclosed herein, are illustrated in Scheme 1.

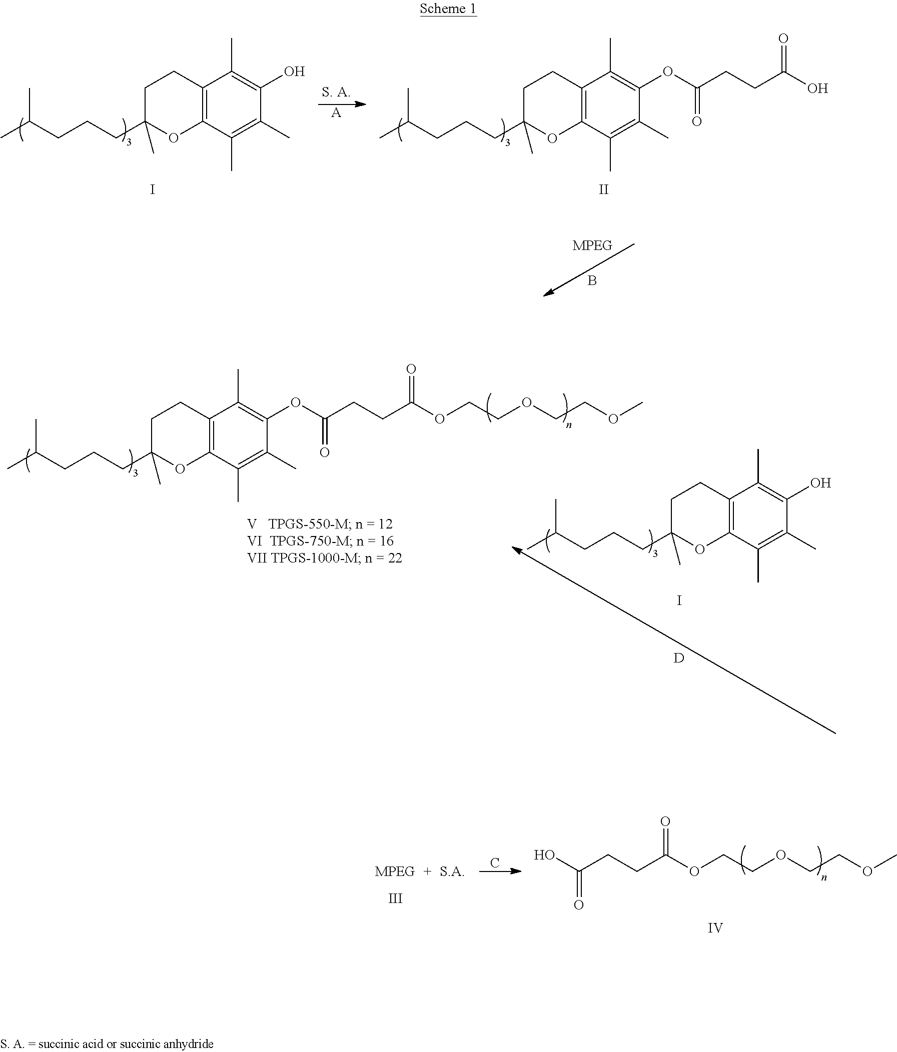

In one embodiment, DL-α-tocopherol may be condensed with succinic anhydride or succinic acid (“S.A.”) under condition A to provide the tocopherol-succinate intermediate II (DL-α-tocopherol succinate). The tocopherol-succinate intermediate may be isolated or may be further condensed with an MPEG under condition B to provide the TPGS-MPEG. Alternatively, MPEG may be condensed with succinic anhydride or succinic acid (“S.A.”) under condition C to form an MPEG-succinate intermediate. The MPEG-succinate intermediate may be condensed with DL-α-tocopherol under condition D to form the TPGS-MPEG.

The condensation or esterification reaction between DL-α-tocopherol and succinic anhydride or succinic acid (S.A.) may be performed under a variety of conditions noted as A. For example, the succinic anhydride may be contacted with DL-α-tocopherol in an aprotic solvent such as toluene, xylenes, ethers such as THF, diethyl ether and dioxane, ethyl acetate, acetone, DMF, N,N-dimethylacetamide, acetonitrile, MEK, MIBK, DMSO, ethyleneglycol dimethylether, hexanes, cyclohexane, pentane, cyclopentane, etc. . . . or mixtures thereof. In one aspect, the solvent is toluene. In one aspect, an inorganic base or an organic base may be added to the reaction mixture containing DL-α-tocopherol and S.A. The inorganic base may be selected from the group consisting of NaHCO₃, Ba(OH)₂, Ca(OH)₂, LiOH, NaOH, KOH, Cs₂CO₃K₂CO₃, LiCO₃, Na₂CO₃ and mixtures thereof. The organic base may be selected from Et₃N, DBU, DBN, and/or in the presence of DMAP. In one variation, the molar ratio of DL-α-tocopherol to S.A. may be about 1:1, 1:1.1, 1:1.2, 1:1.3, 1:1.4 or 1:1.5.

When succinic acid is employed, esterification can be performed using a catalytic amount of an acid as known in the art. In one embodiment, the activation of succinic acid to the corresponding acid halide, such as the acid chloride, may be performed by using a halogenating agent such as SOCl₂, PCl₃, POCl₃, phosgene or phosgene equivalents, optionally with an amine base such as Et₃N, DBU, DBN, pyridine, and/or in the presence of DMAP. Activation may be performed before or during the addition of DL-α-tocopherol. Also, where succinic acid is used instead of succinic anhydride, a higher molar ratio of succinic acid may be employed effectively because of the succinic acid is significantly less expensive. Accordingly, the molar ratio of DL-α-tocopherol to succinic acid may be about 1:1, 1:1.2, 1:1.3, 1:1.5, 1:1.7, 1:1.9 or 1:2. The ratio (wt/wt) of DL-α-tocopherol to the solvent may be about 0.2:1, 0.3:1, 0.4:1, 0.5:1 or about 1:1.

At higher concentration of DL-α-tocopherol, the solution may be rendered homogeneous upon heating and stirring of the reaction mixture. Optionally, a base such as an amine base, including, for example, Et₃N, pyridine, DBN or DBU may be added. In one aspect, the amine is Et₃N. The base may be used in a catalytic amount relative to DL-α-tocopherol, such as about 25 mole %, 15 mole %, 10 mole %, 5 mole %, 3 mole % or less. In one aspect, the base is used in about 25 mole % or less. The reaction may be performed at an elevated temperature, such as about 30 to 90° C., 40 to 80° C., 45 to 75° C., 50 to 70° C., 55 to 65° C., about 60° C., 30 to 50° C., 40 to 60° C., 50 to 70° C., 60 to 80° C. or about 70 to 90° C. In one embodiment, the reaction is performed at an elevated temperature for a sufficient period of time to provide the desired product II (DL-α-tocopherol succinate) such as for less than about 8 hours, 6 hours, 3 hours, 2 hours or about 1 hour.

In one variation, upon the completion of the reaction, water may be added to the reaction mixture, and the product II is then extracted with a solvent such as toluene, diethyl ether or THF. Optionally, the extracts containing the product II may be filtered, such as by filtration on a plug of silica gel or celite. Optionally, the plug of silica gel or celite may be washed with a solvent or solvent mixture such as about 10% to 40% EtOAc/hexane. Where higher product purity is desired, the solvent extracts may be further washed with water or 1N HCl, and then again with water. Extraction procedures may be used where the purity or quality of the starting reagents have lower purity specifications or lower purity profiles. The resulting solvent extracts may be concentrated by distillation under vacuum to provide the product II. Optionally, the product II from the condensation reaction is obtained in sufficient high purity that no filtration and/or no extraction is required; and the solvent is removed by distillation under vacuum to afford a white or semi-white solid. Accordingly, the reaction provides the product II in more than about 95% yield, 97% yield, 98% yield or about 99% yield.

In one embodiment, the product II obtained from the condensation reaction is not further purified or isolated, and the “crude” product II is further condensed with MPEG under condition B, in a one-pot procedure. Using this procedure, removal of the solvent, such as toluene, is not required where the subsequent reaction step also utilizes the same solvent. Such one-pot reaction procedures eliminate the isolation steps, including filtration, washing and solvent removal steps, and provide significantly shorter overall reaction cycle times and increase product throughput. Accordingly, the product II is then contacted with MPEG (polyethylene glycol monomethylether) under conditions as described herein to form the product V, VI or VII without any intermediate purification or isolation steps.

Depending on the desired product, the MPEG employed as the reagent in the condensation reaction may have different molecular weights, where the MPEG may be selected from any MPEG between MPEG-300 and MPEG-2000. More specifically, the choice would be MPEG-550, MPEG-750, or MPEG-1000.

In one variation, the solvent used in the condensation reaction may be an aprotic solvent such as toluene, xylenes, ethers such as THF, diethyl ether and dioxane, ethyl acetate, acetone, DMF, N,N-dimethylacetamide, acetonitrile, MEK, MIBK, DMSO, ethyleneglycol dimethylether, hexanes, cyclohexane, pentane, cyclopentane, etc. . . . or mixtures thereof. In one aspect, the solvent is toluene.

The mole ratio of II to the MPEG may be about 1:1, 1:1.01, 1:1.02, 1:1.04, 1:1.05, 1:1.1, or about 1:1.2. In one variation, the mole ratio of II to MPEG may be about 1:1.05. Optionally, a catalytic amount of an acid, such as Fe³⁺ (or Zr or Al)/Montmorillonite clay catalyst, sulfuric acid, dry HCl, Amberlyst, Nafion-H, SiO₂—Al₂O₃, p-TsOH, etc. . . . The mole % of the acid relative to II may be used in an amount of about 15 mole %, 10 mole %, 5 mole %, 3 mole %, or 1 mole % or less. In one variation, the acid is p-TsOH monohydrate in about 10 mole %, 5 mole % or less.

The reaction mixture comprising II, MPEG and acid in a solvent, such as toluene, may be heated at an elevated temperature, such as to reflux, to azeotropically remove water from the reaction mixture. Such azeotropic removal of water may be performed using a Dean-Stark trap or an equivalent distillation set-up to remove water. The reaction may be heated for at least 2 hours, 3 hours, 5 hours or more, until II is completely consumed. Where II is not consumed over the reaction times, optionally, the reaction mixture may be cooled below refluxing temperatures, such as about 100° C., 90° C. or 75° C. or less, and an additional amount of MPEG, such as about 5 mole % relative to the original amount of II, may be added. The resulting mixture may be re-heated to reflux until the starting material II is found to be completely or substantially consumed.

Upon completion of the reaction, the resulting mixture is cooled to room temperature and the solvent was removed by distillation under vacuum. Optionally, the resulting cooled mixture is filtered over a plug or a pad of silica gel or celite to remove dark tars or insoluble components before removal of solvent by vacuum distillation. Also optionally, an aqueous NaHCO₃ solution is added to the resulting cooled mixture and the organic product is extracted with a solvent, such as toluene, THF or CH₂Cl₂. The combined extracts may be dried by distillation in vacuum of dried over anhydrous Na₂SO₄. The product V, VI or VII may be isolated from the organic extracts by distillation in vacuum to provide the desired product as a waxy solid. The product obtained provides HPLC, ¹H NMR, ¹³C NMR and M.S. spectrum consistent with the desired product.

In one particular embodiment, TPGS variants with MPEG molecular weights of approximately 550 (n=ca. 12), 750 (n=ca. 17) and 1000 (n=ca. 23) were synthesized via the 2-step route outlined in Scheme 2. Under optimized conditions on a laboratory scale of <10 g, as illustrated for TPGS-750-M, each of the two steps affords a nearly quantitative yield of the desired product. Ring opening of succinic anhydride (1.5 equiv) by α-tocopherol in warm toluene (0.5 M) takes place smoothly in five hours. The resulting acid is then put through a standard workup and filtration through silica gel to give known white solid H. See Nakamura, T.; Kijima, S. α-Tocopheryl acid succinate. G.B. Patent 1,114,150, May 15, 1968. Treatment of ester H with MPEG-750 in the usual way (cat. TsOH, toluene, heat, Dean Stark trap) gave the desired, previously unknown amphiphile VI as a waxy solid. This sequence could be smoothly scaled to >150 g, with comparable yields for each step (97% and 98%, respectively). In a similar fashion, both TPGS-600 and TPGS-550-M were prepared as viscous liquid materials. All could be stored indefinitely in vials at ambient temperatures.

In one variation, the acid H may be converted into the corresponding activated carboxylic acid derivative IIa, such as the acid chloride, acid bromide, acid iodide, ester or mixed anhydride, for condensation with an MPEG.

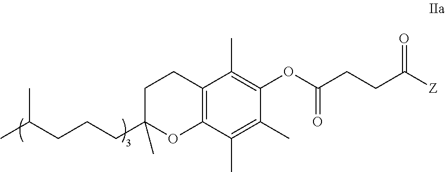

wherein Z is selected from the group consisting of —Cl, —Br, —I and —OR^{o}, wherein R^{o} is selected from the group consisting of C₁₋₃alkyl, —OC(O)C₁₋₆alkyl, —OC(O)CH₂Ph and —OSO₂G where G is C₁₋₆alkyl, aryl or substituted aryl.

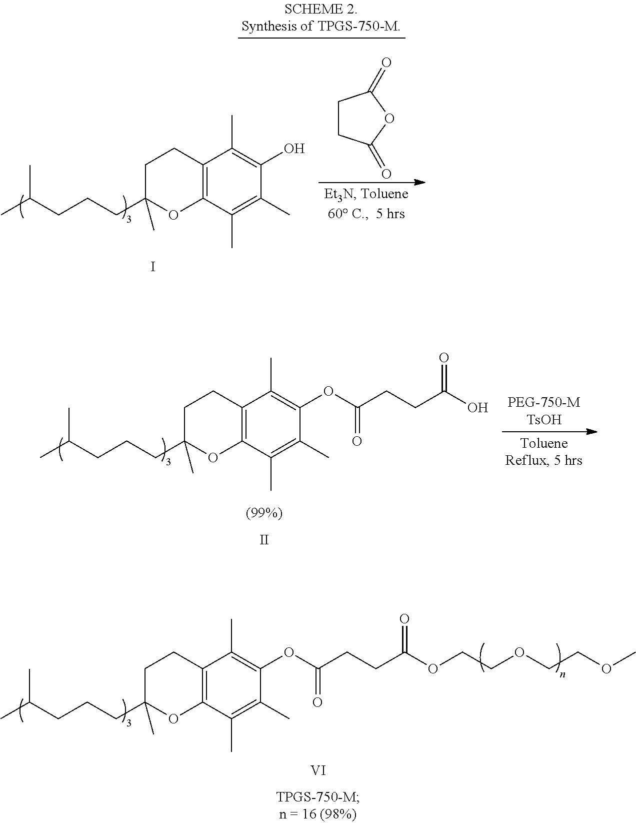

The following embodiments, aspects and variations thereof are exemplary and illustrative are not intended to be limiting in scope.

In one embodiment, using racemic vitamin E, there is provided a racemic compound of the formulae V, VI and VII:

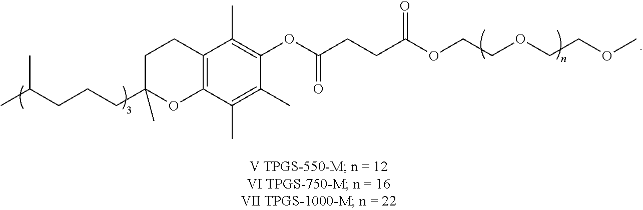

In another embodiment, using racemic vitamin E, there is provided a racemic compound of the formula II:

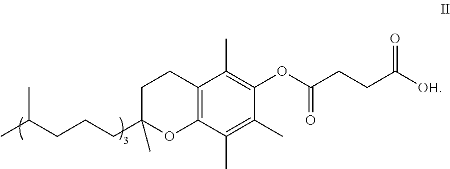

In another embodiment, using racemic vitamin E, there is provided a method for the preparation of a surfactant having the formula V, VI or VII, the method comprising the steps of:

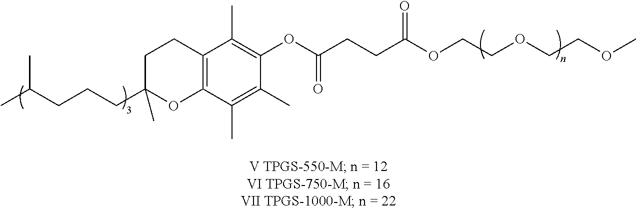

contacting DL-α-tocopherol with succinic anhydride or succinic acid under conditions sufficient to form a compound of the formula II;

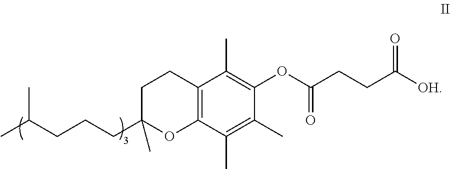

contacting the compound of the formula II with MPEG-550, MPEG-750 or MPEG-1000, at an elevated temperature and under conditions sufficient to form the compound of the formula V, VI or VII, respectively, and isolating the compound of the formula V, VI or VII.

In addition to the exemplary embodiments, aspects and variations described above, further embodiments, aspects and variations will become apparent by reference to the drawings and figures and by examination of the following descriptions.

### DETAILED DESCRIPTION OF THE INVENTION

### Definitions

Unless specifically noted otherwise herein, the definitions of the terms used are standard definitions used in the art of organic synthesis and pharmaceutical sciences. Exemplary embodiments, aspects and variations are illustratived in the figures and drawings, and it is intended that the embodiments, aspects and variations, and the figures and drawings disclosed herein are to be considered illustrative and not limiting.

“DL-α-tocopherol” as used herein refers to the racemic α-tocopherol that may be obtained by synthesis. The racemic α-tocopherol includes all possible enantiomeric and diastereomeric centers, including: 2R, 4′R, 8′R; 2R, 4′R, 8′S; 2R, 4′S, 8′S; 2S, 4′S, 8′S; 2R, 4′S, 8′R; 2S, 4′R, 8′S; 2S, 4′R, 8′R; and 2S, 4′S, 8′R; as shown below.

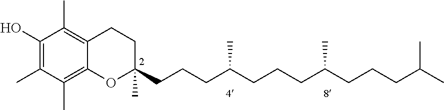

The racemic α-tocopherol that may be employed in the present application also include various different ratios of each of the isomers noted above.

“MPEG” as used herein refers to polyethylene glycol monomethyl ether (PEG monomethyl ether). Suitable polyethylene glycol methyl ethers (MPEG), such as PEG-550-M, PEG-750-M or PEG-1000-M, that are derived from polyethylene glycols (PEG) are commercially available, usually as mixtures of oligomers characterized by an average molecular weight. In one embodiment, polyethylene glycol fragments of the MPEG have an average molecular weight from about 500 to about 1500, and those having an average molecular weight from about 600 to about 900, and those having an average molecular weight of about 750 being particularly preferred. Both linear and branched PEG molecules can be used in the solubilizing agents in the present application. In another embodiment, the PEG fragment of the MPEG has between 5 and 50 subunits. In another embodiment, the PEG fragment of the MPEG has between 16 and 20 subunits. In another embodiment, the PEG of the MPEG has 17 subunits.

Although most sources of MPEG (and PEG) are characterized as a range of compounds based on the number of polyethyleneoxide subunits, narrower ranges are also available (commercially and otherwise) based on a controlled polymerization of ethylene oxide. These more narrowly dispersed MPEGs (and PEGs) are also included in this application, as the routes to the corresponding surfactants fully apply to their use as well.

Each MPEG (and PEG), being a broad range of compounds varying in molecular weight as a function of the number of PEG units, is also subject to peak shaving, where either lower or higher molecular weight components are removed on either or both sides of the central, predominant component (e.g., by chromatographic separation). Such MPEG (or PEG) compositions are also fully amenable to the syntheses of their corresponding new surfactants based on the synthetic routes disclosed herein. Representative ranges, for example, below and above the center for MPEG-550 would be MPEG-450 to MPEG-650; for MPEG-750, a range of MPEG-650 to MPEG-850; and for MPEG-1000, a range of MPEG-850 to MPEG-1200. Various combinations and permutations of two or more MPEGs (and PEGs) could be pre-formed, in any ratio, and subsequently used in the routes to the corresponding mixture of TPGS-MPEG surfactants, thereby resulting in non-Gausian ratios of MPEG-containing surfactants. The chemistry routes as described within this application apply equally well to any and all such mixtures of MPEGs (or PEGs).

A “substituent,” as used herein, means a group that may be used in place of a hydrogen atom in a particular group, such as an alkyl group or an aryl group. Such substituent may include, for example: —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″)═NR″′, —S(O)R′, —S(O)₂R′, —S(O)₂NR′R″, —NRSO₂R′, —CN and —NO₂, —R′, —N₃, —CH(Ph)₂, fluoro(C₁₋₄)alkoxy and fluoro(C₁₋₄)alkyl, in a number ranging from zero to the total number of open valences on the aromatic ring system; and where R′, R″, R″′ and R″″ are preferably independently selected from hydrogen, (C₁₋₈)alkyl and heteroalkyl, unsubstituted aryl and heteroaryl, (unsubstituted aryl)-(C₁₋₄)alkyl, and (unsubstituted aryl)oxy-(C₁₋₄)alkyl. When a compound includes more than one R group, for example, each of the R groups is independently selected as is each R′, R″, R′″ and R″″ group when more than one of these groups are present.

### DESCRIPTION OF THE FIGURE

FIG. 1 illustrates a structural comparison between the various surfactants, including PTS, TPGS-750-M and TPGS (TPGS-1000).

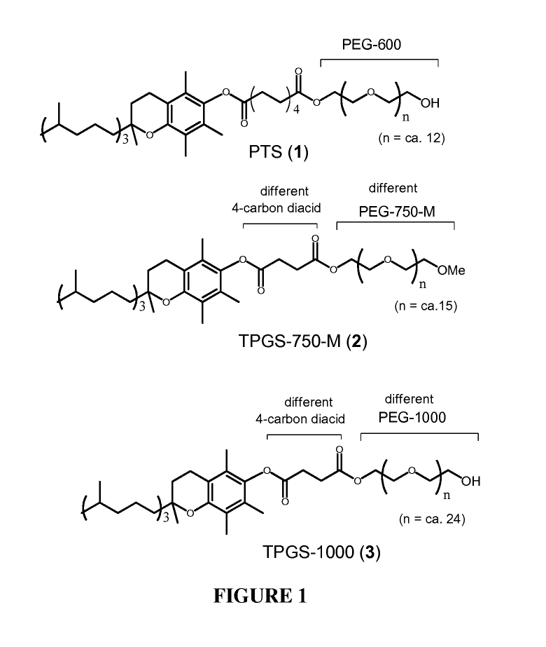

### EXPERIMENTAL

The following procedures may be employed for the preparation of the compounds of the present invention. The starting materials and reagents used in preparing these compounds are either available from commercial suppliers such as the Aldrich Chemical Company (Milwaukee, Wis.), Bachem (Torrance, Calif.), Sigma (St. Louis, Mo.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

DL-α-Tocopherol succinate (II); <10 g scale. To a solution of DL-α-tocopherol (4.30 g, 10.00 mmol) and succinic anhydride (1.50 g, 15.00 mmol) in toluene (20 mL), Et₃N (0.35 mL, 2.50 mmol) was added at 22° C. with stirring, and the stirring was continued at 60° C. for 5 h. Water was added to the reaction mixture, which was then extracted with CH₂Cl₂. The combined organic layers were washed with 1N HCl (3×50 mL), water (2×30 mL), dried over anhydrous Na₂SO₄, and concentrated in vacuo affording a yellow liquid, which was purified by flash column chromatography on silica gel eluting with a 10% EtOAC/hexane to 35% EtOAC/hexanes gradient to afford DL-α-tocopherol succinate (5.25 g, 99%) as a white solid, mp 68-71° C., lit mp 64-67° C.; IR (neat): 2926, 1757, 1714, 1576, 1463, 1455, 1415, 1377, 1251, 1224, 1151, 1110, 1078, 926 cm⁻¹; ¹H NMR (400 MHz, CDCl₃): δ 2.94 (t, J=6.8 Hz, 2H), 2.84 (t, J=6.8 Hz, 2H), 2.59 (t, J=6.8 Hz, 2H), 2.09 (s, 3H), 2.02 (s, 3H), 1.98 (s, 3H), 1.85-1.71 (m, 2H), 1.56-1.50 (m, 3H), 1.43-1.05 (m, 21H), 0.88-0.84 (m, 12H); ¹³C NMR (100 MHz, CDCl₃): δ 178.6, 171.0, 149.7, 140.7, 126.9, 125.1, 123.2, 117.6, 75.2, 39.6, 37.8, 37.7, 37.6, 37.5, 33.0, 32.9, 31.3, 29.2, 28.8, 28.2, 25.0, 24.6, 24.0, 22.9, 22.8, 21.2, 20.8, 19.95, 19.88, 13.0, 12.2, 12.0; MS (ESI): m/z 554 (M+Na); HRMS (ESI) calcd for C₃₃H₅₄O₅Na [M+Na]⁺=553.3869. found 553.3876.

TPGS-750-M (VI). A mixture containing DL-α-tocopherol succinate (2.97 g, 5.60 mmol), polyethylene glycol monomethylether-750 (4.00 g, 5.33 mmol) and p-TsOH (0.15 g, 0.79 mmol) in toluene (20 mL) was refluxed for 5 h using a Dean-Stark trap. After cooling to rt, the mixture was poured into saturated aqueous NaHCO₃ solution and extracted with CH₂Cl₂. The combined organic layers were washed with saturated NaHCO₃ (3×50 mL), brine (2×30 mL), dried over anhydrous Na₂SO₄ and concentrated in vacuo to afford the title compound (6.60 g, 98%) as a waxy solid. IR (neat): 2888, 1755, 1739, 1465, 1414, 1346, 1281, 1245, 1202, 1109, 947, 845 cm⁻¹; ¹H NMR (400 MHz, CDCl₃): δ 4.28-4.26 (m, 2H), 3.71-3.54 (m, PEG), 3.38 (s, 3H), 2.93 (t, J=7.2 Hz, 2H), 2.79 (t, J=7.2 Hz, 2H), 2.58 (t, J=6.8 Hz, 2H), 2.08 (s, 3H), 2.01 (s, 3H), 1.97 (s, 3H), 1.84-1.70 (m, 2H), 1.55-1.04 (m, 24H), 0.87-0.83 (m, 12H); ¹³C NMR (100 MHz, CDCl₃): δ 172.2, 170.9, 149.5, 140.6, 126.7, 125.0, 123.0, 117.4, 94.5, 75.1, 72.0, 70.64, 70.56, 69.1, 64.0, 59.0, 39.4, 37.6, 37.5, 37.4, 37.3, 32.8, 32.7, 31.1, 29.2, 28.9, 28.0, 24.8, 24.5, 22.8, 22.7, 21.1, 20.6, 19.8, 19.7, 13.0, 12.1, 11.8; MS (ESI): m/z 1272 (M+Na).

DL-α-Tocopherol succinate (II); >150 g scale. 2,5,7,8-Tetramethyl-2-(4,8,12-trimethyltridecyl)chroman-6-ol (DL-α-Tocopherol, 66.4 g, 154.1 mmol) and methylene chloride (300 mL) were charged under nitrogen into a 1 L single necked round bottom flask which had been oven-dried and cooled under vacuum. Succinic anhydride (23.1 g, 231 mmol) was added to the clear yellow solution followed by the addition of 4-dimethylaminopyridine (9.4 g, 77.1 mmol) and finally triethylamine (21.5 mL, 154 mmol). The reaction mixture was stirred at 23° C. overnight during which time the reaction mixture became a dark purplish solution. HPLC and TLC (3:7 EtOAc:hexanes, R_{f}=0.3) indicated the reaction was complete. The reaction mixture was poured into a 1 L separatory funnel and the flask rinsed with methylene chloride (300 mL). The organic layer was washed with 1M HCl (160 mL) (×3), water (100 mL) (×2), and saturated aqueous sodium chloride solution (250 mL). The organic layer was dried over sodium sulfate, filtered and the solvent removed in vacuo affording a dark, viscous oil. The oil was poured onto a pad of silica gel (600 g in a 1.2 L filter funnel) and then eluted first with methylene chloride (1.5 L) (to remove impurity) followed by elution with 1:1 EtOAc:hexane (3 L). Concentration of the solvent in vacuo followed by storage under high vacuum overnight affords 82.6 g of a faintly yellow semi-solid containing 4 wt. % EtOAc (79.3 g actual, 96.9%). NMR (CDCl₃) was consistent with the desired product. Used as is for the next reaction.

TPGS-750-M (VI). 4-oxo-4-{[2,5,7,8-tetramethyl-2-(4,8,12-trimethyltridecyl)-3,4-dihydro-2H-chromen-6-yl]oxy}butanoic acid (79.3 g, 149 mmol) was dissolved in toluene (560 mL, 5.3 mol) in a 1 L 3-necked round bottom flask under a stream of nitrogen. MPEG-750 (105 g, 142 mmol) was added to the reaction mixture followed by the addition of p-toluenesulfonic acid monohydrate (3.01 g, 15.8 mmol) which caused a slight lightening of the solution. The flask was fitted with a Dean-Stark trap (receiver filled with toluene) and a condenser. The reaction mixture was heated to reflux for 5 hours. HPLC indicates that SM still remains. The reaction mixture was cooled to room temperature, additional MPEG 750 (5.00 g, 6.78 mmol) was added, and the reaction was heated to reflux for an additional 5 hours. HPLC indicated that almost all of the SM was gone. The reaction mixture was cooled to room temperature and concentrated on a rotary evaporator to afford a viscous dark brown oil. The oil was passed through a pad of basic aluminum oxide (600 g in a 1.2 L filter funnel) eluting with methylene chloride (3 L). The solvent was removed in vacuo to afford a faintly yellow waxy solid. The material is placed under high vacuum keeping the material at 50° C. (the waxy solid liquefies at this temperature) until removal of the residual toluene and methylene chloride was complete. After cooling and re-solidification, 174 g (98.2%) of material was obtained that is identical in all aspects (HPLC, ¹H NMR, ¹³C NMR) with the sample prepared on a smaller scale.

TPGS surfactants, including TPGS-550-M, TPGS-750-M and TPGS-1000-M may be prepared according to representative procedures and reaction conditions disclosed in the present application, as noted in the Tables 1-2:

TABLE 1

| | | Results |
|---|---|---|
| | Reaction Conditions | (% Conversion, |
| Entry | Condition A: | HPLC) |
| 1 | Succinic anhydride (1.5 mole equiv.) | >95-99% |
| | Toluene; Et₃N (25 mole %); 60° C., 5 hrs | |
| 2 | Succinic anhydride (1.3 mole equiv.) | >95-99% |
| | Toluene; Et₃N (25 mole %); 60° C., 5 hrs | |
| 3 | Succinic anhydride (1.2 mole equiv.) | >95-99% |
| | Toluene; Et₃N (25 mole %); 60° C., 5 hrs | |
| 4 | Succinic anhydride (1.5 mole equiv.) | >95-99% |
| | Toluene; Et₃N (25 mole %); 60° C., 5 hrs | |
| 5 | Succinic anhydride (1.5 mole equiv.) | >95-99% |
| | Toluene; Et₃N (20 mole %); 60° C., 5 hrs | |
| 6 | Succinic anhydride (1.5 mole equiv.) | >95-99% |
| | Toluene; Et₃N (15 mole %); 60° C., 5 hrs | |
| 7 | Succinic anhydride (1.5 mole equiv.) | >95-99% |
| | Xylenes; Et₃N (25 mole %); 60° C., 5 hrs | |
| 8 | Succinic anhydride (1.3 mole equiv.) | >95-99% |
| | Xylenes; Et₃N (25 mole %); 60° C., 5 hrs | |
| 9 | Succinic anhydride (1.2 mole equiv.) | >95-99% |
| | Xylenes; Et₃N (25 mole %); 60° C., 5 hrs | |
| 10 | Succinic anhydride (1.5 mole equiv.) | >95-99% |
| | Xylenes; Et₃N (25 mole %); 70° C., 3 hrs | |
| 11 | Succinic anhydride (1.3 mole equiv.) | >95-99% |
| | Xylenes; Et₃N (25 mole %); 70° C., 3 hrs | |
| 12 | Succinic anhydride (1.2 mole equiv.) | >95-99% |
| | Xylenes; Et₃N (25 mole %); 70° C., 3 hrs | |
| 13 | Succinic anhydride (1.2 mole equiv.) | >95-99% |
| | Xylenes; Et₃N (25 mole %); 70° C., 3 hrs | |
| 14 | Succinic anhydride (1.2 mole equiv.) | >95-99% |
| | Xylenes; Et₃N (20 mole %); 70° C., 3 hrs | |
| 15 | Succinic anhydride (1.2 mole equiv.) | >95-99% |
| | Xylenes; Et₃N (15 mole %); 70° C., 3 hrs | |
| 16 | Succinic acid (1.5 mole equiv.) | >95-99% |
| | Toluene; Et₃N (25 mole %); 60° C., 5 hrs | |
| 17 | Succinic acid (1.3 mole equiv.) | >95-99% |
| | Toluene; Et₃N (25 mole %); 60° C., 5 hrs | |
| 18 | Succinic acid (1.2 mole equiv.) | >95-99% |
| | Toluene; Et₃N (25 mole %); 60° C., 5 hrs | |
| 19 | Succinic acid (1.5 mole equiv.) | >95-99% |
| | Toluene; Et₃N (25 mole %); 60° C., 5 hrs | |
| 20 | Succinic acid (1.5 mole equiv.) | >95-99% |
| | Toluene; Et₃N (20 mole %); 60° C., 5 hrs | |
| 21 | Succinic acid (1.5 mole equiv.) | >95-99% |
| | Toluene; Et₃N (15 mole %); 60° C., 5 hrs | |
| 22 | Succinic acid (1.5 mole equiv.) | >95-99% |
| | Xylenes; Et₃N (25 mole %); 60° C., 5 hrs | |
| 23 | Succinic acid (1.3 mole equiv.) | >95-99% |
| | Xylenes; Et₃N (25 mole %); 60° C., 5 hrs | |
| 24 | Succinic acid (1.5 mole equiv.) | >95-99% |
| | Xylenes; Et₃N (25 mole %); 70° C., 3 hrs | |
| 25 | Succinic acid (1.2 mole equiv.) | >95-99% |
| | Xylenes; Et₃N (25 mole %); 60° C., 5 hrs | |
| 26 | Succinic acid (1.3 mole equiv.) | >95-99% |
| | Xylenes; Et₃N (25 mole %); 70° C., 3 hrs | |
| 27 | Succinic acid (1.2 mole equiv.) | >95-99% |
| | Xylenes; Et₃N (25 mole %); 70° C., 3 hrs | |
| 28 | Succinic acid (1.2 mole equiv.) | >95-99% |
| | Xylenes; Et₃N (25 mole %); 70° C., 3 hrs | |
| 29 | Succinic acid (1.2 mole equiv.) | >95-99% |
| | Xylenes; Et₃N (20 mole %); 70° C., 3 hrs | |
| 30 | Succinic acid (1.2 mole equiv.) | >95-99% |
| | Xylenes; Et₃N (15 mole %); 70° C., 3 hrs | |
| 31 | Succinic acid (1.5 mole equiv.); SOCl₂ | >95-99% |
| | (1 mole equiv.) | |
| | Toluene; Et₃N (25 mole %); 60° C., 5 hrs | |
| 32 | Succinic acid (1.3 mole equiv.); SOCl₂ | >95-99% |
| | (1 mole equiv.) | |
| | Toluene; Et₃N (25 mole %); 60° C., 5 hrs | |
| 33 | Succinic acid (1.2 mole equiv.); SOCl₂ | >95-99% |
| | (1 mole equiv.) | |
| | Toluene; Et₃N (25 mole %); 60° C., 5 hrs | |

TABLE 2

| | | Results |
|---|---|---|
| | Reaction Conditions | (% Conversion, |
| Entry | Condition B: | HPLC) |
| 1 | MPEG-600 (1.7 mole equiv.) | >95-98% |
| | Toluene (reflux), TsOH (0.15 mole equiv.) | |
| 2 | MPEG-600 (1.5 mole equiv.) | >95-98% |
| | Toluene (reflux), TsOH (0.15 mole equiv.) | |
| 3 | MPEG-600 (1.3 mole equiv.) | >95-98% |
| | Toluene (reflux), TsOH (0.15 mole equiv.) | |
| 4 | MPEG-600 (1.2 mole equiv.) | >95-98% |
| | Toluene (reflux), TsOH (0.15 mole equiv.) | |
| 5 | MPEG-600 (1.7 mole equiv.) | >95-98% |
| | Toluene (reflux), TsOH (0.2 mole equiv.) | |
| 6 | MPEG-600 (1.5 mole equiv.) | >95-98% |
| | Toluene (reflux), TsOH (0.17 mole equiv.) | |
| 7 | MPEG-600 (1.3 mole equiv.) | >95-98% |
| | Toluene (reflux), TsOH (0.13 mole equiv.) | |
| 8 | MPEG-600 (1.2 mole equiv.) | >95-98% |
| | Toluene (reflux), TsOH (0.12 mole equiv.) | |
| 9 | MPEG-600 (1.2 mole equiv.) | >95-98% |
| | Toluene (reflux), TsOH (0.1 mole equiv.) | |
| 10 | MPEG-600 (1.7 mole equiv.) | >95-98% |
| | Xylenes (105° C.), TsOH (0.15 mole equiv.) | |
| 11 | MPEG-600 (1.5 mole equiv.) | >95-98% |
| | Xylenes (105° C.), TsOH (0.15 mole equiv.) | |
| 12 | MPEG-600 (1.3 mole equiv.) | >95-98% |
| | Xylenes (105° C.), TsOH (0.15 mole equiv.) | |
| 13 | MPEG-600 (1.2 mole equiv.) | >95-98% |
| | Xylenes (105° C.), TsOH (0.15 mole equiv.) | |
| 14 | MPEG-600 (1.7 mole equiv.) | >95-98% |
| | Xylenes (105° C.), TsOH (0.2 mole equiv.) | |
| 15 | MPEG-600 (1.5 mole equiv.) | >95-98% |
| | Xylenes (105° C.), TsOH (0.17 mole equiv.) | |
| 16 | MPEG-600 (1.3 mole equiv.) | >95-98% |
| | Xylenes (105° C.), TsOH (0.13 mole equiv.) | |
| 17 | MPEG-600 (1.2 mole equiv.) | >95-98% |
| | Xylenes (105° C.), TsOH (0.12 mole equiv.) | |
| 18 | MPEG-600 (1.2 mole equiv.) | >95-98% |
| | Xylenes (105° C.), TsOH (0.1 mole equiv.) | |
| 19 | MPEG-600 (1.7 mole equiv.) | >95-98% |
| | Toluene (reflux), TsOH (0.15 mole equiv.) | |
| 20 | MPEG-750 (1.7 mole equiv.) | >95-98% |
| | Toluene (reflux), TsOH (0.15 mole equiv.) | |
| 21 | MPEG-750 (1.5 mole equiv.) | >95-98% |
| | Toluene (reflux), TsOH (0.15 mole equiv.) | |
| 23 | MPEG-750 (1.3 mole equiv.) | >95-98% |
| | Toluene (reflux), TsOH (0.15 mole equiv.) | |
| 24 | MPEG-750 (1.2 mole equiv.) | >95-98% |
| | Toluene (reflux), TsOH (0.15 mole equiv.) | |
| 25 | MPEG-750 (1.7 mole equiv.) | >95-98% |
| | Toluene (reflux), TsOH (0.2 mole equiv.) | |
| 26 | MPEG-750 (1.5 mole equiv.) | >95-98% |
| | Toluene (reflux), TsOH (0.17 mole equiv.) | |
| 27 | MPEG-750 (1.3 mole equiv.) | >95-98% |
| | Toluene (reflux), TsOH (0.13 mole equiv.) | |
| 28 | MPEG-750 (1.2 mole equiv.) | >95-98% |
| | Toluene (reflux), TsOH (0.12 mole equiv.) | |
| 29 | MPEG-750 (1.2 mole equiv.) | >95-98% |
| | Toluene (reflux), TsOH (0.1 mole equiv.) | |
| 30 | MPEG-750 (1.7 mole equiv.) | >95-98% |
| | Xylenes (105° C.), TsOH (0.15 mole equiv.) | |
| 31 | MPEG-750 (1.5 mole equiv.) | >95-98% |
| | Xylenes (105° C.), TsOH (0.15 mole equiv.) | |
| 32 | MPEG-750 (1.3 mole equiv.) | >95-98% |
| | Xylenes (105° C.), TsOH (0.15 mole equiv.) | |
| 33 | MPEG-750 (1.2 mole equiv.) | >95-98% |
| | Xylenes (105° C.), TsOH (0.15 mole equiv.) | |
| 34 | MPEG-750 (1.7 mole equiv.) | >95-98% |
| | Xylenes (105° C.), TsOH (0.2 mole equiv.) | |
| 35 | MPEG-750 (1.5 mole equiv.) | >95-98% |
| | Xylenes (105° C.), TsOH (0.17 mole equiv.) | |
| 36 | MPEG-750 (1.3 mole equiv.) | >95-98% |
| | Xylenes (105° C.), TsOH (0.13 mole equiv.) | |
| 37 | MPEG-750 (1.2 mole equiv.) | >95-98% |
| | Xylenes (105° C.), TsOH (0.12 mole equiv.) | |
| 38 | MPEG-750 (1.2 mole equiv.) | >95-98% |
| | Xylenes (105° C.), TsOH (0.1 mole equiv.) | |
| 39 | MPEG-750 (1.7 mole equiv.) | >95-98% |
| | Toluene (reflux), TsOH (0.15 mole equiv.) | |
| 40 | MPEG-1000 (1.5 mole equiv.) | >95-98% |
| | Toluene (reflux), TsOH (0.15 mole equiv.) | |
| 41 | MPEG-1000 (1.3 mole equiv.) | >95-98% |
| | Toluene (reflux), TsOH (0.15 mole equiv.) | |
| 42 | MPEG-1000 (1.2 mole equiv.) | >95-98% |
| | Toluene (reflux), TsOH (0.15 mole equiv.) | |
| 43 | MPEG-1000 (1.7 mole equiv.) | >95-98% |
| | Toluene (reflux), TsOH (0.2 mole equiv.) | |
| 44 | MPEG-1000 (1.5 mole equiv.) | >95-98% |
| | Toluene (reflux), TsOH (0.17 mole equiv.) | |
| 45 | MPEG-1000 (1.3 mole equiv.) | >95-98% |
| | Toluene (reflux), TsOH (0.13 mole equiv.) | |
| 46 | MPEG-1000 (1.2 mole equiv.) | >95-98% |
| | Toluene (reflux), TsOH (0.12 mole equiv.) | |
| 47 | MPEG-1000 (1.2 mole equiv.) | >95-98% |
| | Toluene (reflux), TsOH (0.1 mole equiv.) | |
| 48 | MPEG-1000 (1.7 mole equiv.) | >95-98% |
| | Xylenes (105° C.), TsOH (0.15 mole equiv.) | |
| 49 | MPEG-1000 (1.5 mole equiv.) | >95-98% |
| | Xylenes (105° C.), TsOH (0.15 mole equiv.) | |
| 50 | MPEG-1000 (1.3 mole equiv.) | >95-98% |
| | Xylenes (105° C.), TsOH (0.15 mole equiv.) | |
| 51 | MPEG-1000 (1.2 mole equiv.) | >95-98% |
| | Xylenes (105° C.), TsOH (0.15 mole equiv.) | |
| 52 | MPEG-1000 (1.7 mole equiv.) | >95-98% |
| | Xylenes (105° C.), TsOH (0.2 mole equiv.) | |
| 53 | MPEG-1000 (1.5 mole equiv.) | >95-98% |
| | Xylenes (105° C.), TsOH (0.17 mole equiv.) | |
| 54 | MPEG-1000 (1.3 mole equiv.) | >95-98% |
| | Xylenes (105° C.), TsOH (0.13 mole equiv.) | |
| 55 | MPEG-1000 (1.2 mole equiv.) | >95-98% |
| | Xylenes (105° C.), TsOH (0.12 mole equiv.) | |

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

The entire disclosures of all documents cited throughout this application are incorporated herein by reference.

What is claimed is:

1. A racemic compound of the formula VIII:

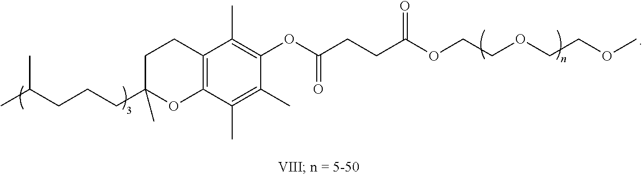

2. The racemic compound of claim 1, wherein the compound is of the formula V, VI or VII:

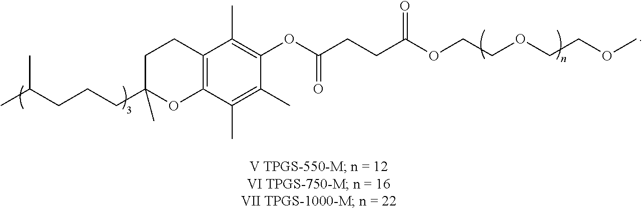

3. The racemic compound of claim 2, wherein the compound is of the formula VI:

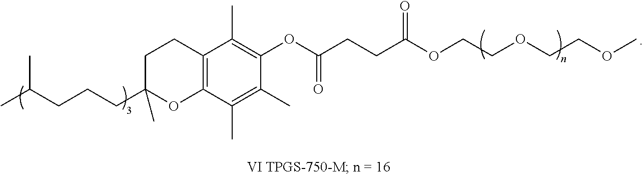

4. A racemic compound of the formula IIa: 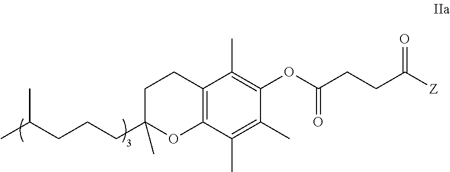
wherein Z is selected from the group consisting of —OH, —Cl, —Br, —I and —OR^{o}, wherein R^{o} is selected from the group consisting of C₁₋₃alkyl, —OC(O)C₁₋₆alkyl and —OC(O)CH₂Ph, and OSO₂G where G is C₁₋₆alkyl, aryl or substituted aryl.

5. A method for the preparation of a surfactant having the formula V, VI or VII, the method comprising the steps of: 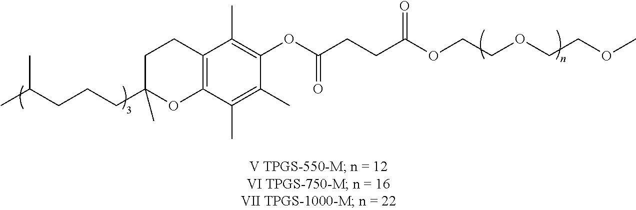
contacting DL-α-tocopherol with succinic anhydride or succinic acid under conditions sufficient to form a compound of the formula II; 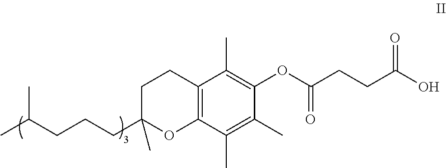
contacting the compound of the formula II with MPEG-550, MPEG-750 or MPEG-1000, at an elevated temperature and under conditions sufficient to form the compound of the formula V, VI or VII, respectively, and isolating the compound of the formula V, VI or VII.

6. The method of claim 5, wherein the step of contacting DL-α-tocopherol with succinic anhydride further comprising a base at an elevated temperature to form the compound of the formula II.

7. The method of claim 6, wherein the base is an organic base comprising of Et₃N with or without catalytic DMAP.

8. The method of claim 5, wherein the ratio of DL-α-tocopherol to succinic anhydride is about 1:1 to 1:1.5.

9. The method of claim 6, wherein the step of contacting DL-α-tocopherol with succinic anhydride and a base is performed in toluene at about 45° C. to 75° C.

10. The method of claim 5, wherein contacting the compound of the formula II with MPEG-550, MPEG-750 or MPEG-1000, is performed in refluxing toluene to remove water.

11. The method of claim 10, wherein the compound of the formula II and MPEG-550, MPEG-750 or MPEG-1000 is further contacted with p-TsOH.

12. The method of claim 5, wherein the compound of formula II is obtained without further isolation and the subsequent step to form the compound of the formula V, VI or VII is performed in a single reaction vessel.

13. A method for the preparation of a surfactant having the formula V, VI or VII, the method comprising the steps of: 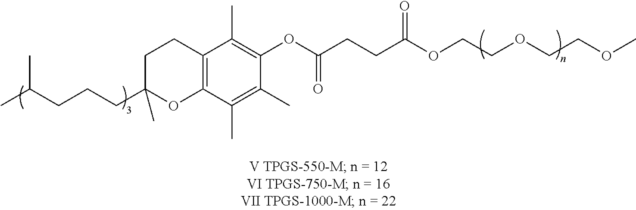
contacting MPEG-550, MPEG 750 or MPEG-1000 with succinic anhydride or succinic acid under conditions sufficient to for a compound of the formulae: 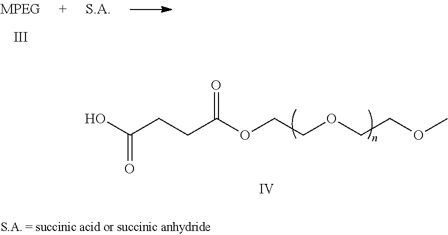
and contacting the compound of the formula IV with DL-α-tocopherol under conditions sufficient to form the compound of the formula V, VI or VII.

14. The method of claim 13, wherein S.A. is succinic acid.

15. The method of claim 13, wherein the step of contacting the compound of formulae IV with DL-α-tocopherol is performed in refluxing toluene with the azeotropic removal of water.

16. A method for preparing TPGS-750-M according to the following two steps: [chemical structure]
